(19) United States
(12) Patent Application Publication
(10) Pub. No.: US 2007/0129437 A1
(43) Pub. Date: Jun. 7, 2007

(54) Process for preparing simvastatin and intermediates thereof

(76) Inventor: Ferenc Korodi, Debrecen (HU)

Correspondence Address: KENYON & KENYON LLP, ONE BROADWAY, NEW YORK, NY, 10004, US

(21) Appl. No.: 11/521,595

(22) Filed: Sep. 14, 2006

Related U.S. Application Data
(60) Provisional application No. 60/742,541, filed on Dec. 6, 2005.

(51) Int. Cl. A61K 31/22; C07C 231/02

(52) U.S. Cl. 514/548; 560/250

(57) ABSTRACT

Novel processes for the preparation of simvastatin and intermediates of such processes. Preferred embodiments include the preparation of lovastatin amides, protected lovastatin amide derivatives, simvastatin dihydroxy acid amide derivatives, alkali salts, simvastatin dihydroxy acids, simvastatin ammonium salts, and simvastatin.

This application claims the benefit of U.S. Provisional Patent Applications Ser. Nos. 60/717,006, filed Sep. 13, 2005, and 60/742,541, filed Dec. 6, 2005, which are incorporated herein by reference, in their entirety.

### FIELD OF INVENTION

The invention relates to processes for preparing simvastatin and intermediates of such processes.

### BACKGROUND OF THE INVENTION

Simvastatin, marketed under the name ZOCOR® by Merck & Co., is a lipid-lowering agent. After oral ingestion, it is believed that simvastatin, an inactive lactone, is hydrolyzed to the corresponding 3,5-dihydroxy acid form, which then inhibits the enzyme 3-hydroxy-3-methylglutaryl-coenzyme A (HMG-CoA) reductase. This enzyme is believed to catalyze the conversion of HMG-CoA to mevalonate, an early rate-limiting step in the biosynthesis of cholesterol.

Simvastatin is also known as butanoic acid, 2,2-dimethyl-1,2,3,7,8,8a-hexahydro-3,7-dimethyl-8-[2-(tetrahydro-4-hydroxy-6-oxo-2H-pyran-2-yl)-ethyl]-1-naphthalenyl ester, [1S-[1(alpha),3(alpha),7(beta),8(beta)(2S*,4S*),-8(alpha)(beta)]. Simvastatin (m.w. 418.57) has the structure represented in formula I below.

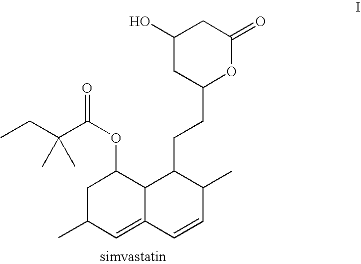

Simvastatin can be synthetically prepared from the fermentation product lovastatin, shown in formula II, by “methylation” processes.

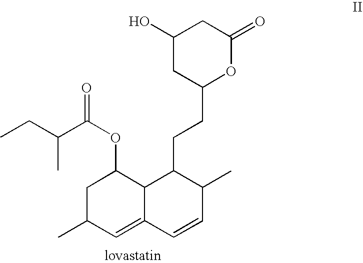

For example, U.S. Pat. No. 4,582,915 describes a process for preparing simvastatin by first converting lovastatin to an alkali metal salt, preferably the potassium salt, of the dihydroxycarboxylate, then methylating the 2-methylbutyryloxy group at the C2-position.

Acidity of the α-protons of the 3,5-dihydroxyheptanoic acid moiety can be decreased by formation of a lovastatin hydroxy acid amide. This dihydroxy amide derivative can be methylated without further protection, as disclosed in U.S. Pat. No. 5,763,646, or after protection of the 1,3-diol moiety by (1) tert-butyldimethylsilylation, as disclosed in U.S. Pat. No. 4,820,850; (2) the formation of phenylboronic acid derivatives, as disclosed in U.S. Pat. No. 5,393,893; (3) the formation of acetonides as disclosed in U.S. Pat. No. 6,100,407; or (4) protection using hexamethyldisilazane (HMDS), as disclosed in U.S. Pat. No. 6,472,542.

The above processes, however, suffer from several disadvantages. Thus, there is a continuing need for processes for preparing simvastatin.

### SUMMARY OF THE INVENTION

In one aspect, the present invention provides a process for the preparation of simvastatin and intermediates useful in making simvastatin.

In one embodiment, the invention relates to a process for the preparation of lovastatin amide of formula III, 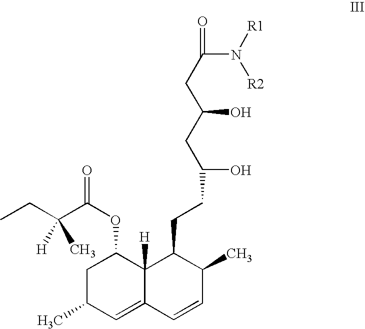
which includes:
(a) combining a lovastatin compound selected from the group consisting of

(i) lovastatin of formula II,

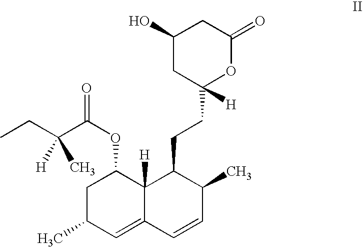

(ii) lovastatin acid of formula VII,

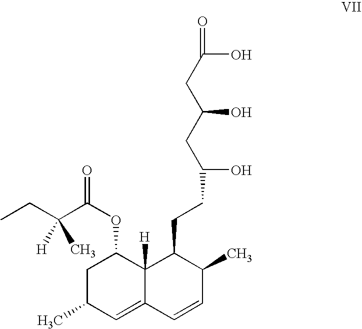

(iii) a salt of lovastatin of formula II,

(iv) a salt of lovastatin acid of formula VII, and

(v) mixtures thereof,

an inert organic solvent, and an amine of formula HNR¹R² to obtain a first reaction mixture; and

(b) obtaining a second reaction mixture comprising the lovastatin amide of formula III by maintaining the first reaction mixture at a temperature and for a period of time sufficient to convert substantially all of the lovastatin compound to the lovastatin amide of formula III.

In such a process, the molar ratio of the amine of formula HNR¹R² to the lovastatin compound is no more than about 1.5, and R¹ and R² are independently selected from hydrogen, straight or branched C₂₋₈ alkyl, aryl, aryalkyl, and C₃₋₈ cycloalkyl groups, or together form a ring optionally containing a heteroatom.

The period of time can be, e.g., from about 3 hours to about 5 hours and the temperature can be from about 60° C. to about 120° C. The amine of formula HNR¹R² can be, e.g., selected from the group consisting of n-butylamine, diethylamine, cyclohexylamine, morpholine, benzylamine and mixtures thereof, e.g., cyclohexylamine, benzylamine or mixtures thereof. In certain embodiments, the molar ratio of the amine of formula HNR¹R² to the lovastatin compound is no more than about 1.2, or from about 1 to about 1.2.

The lovastatin compound can be, e.g., an ammonium salt of formula VIII.

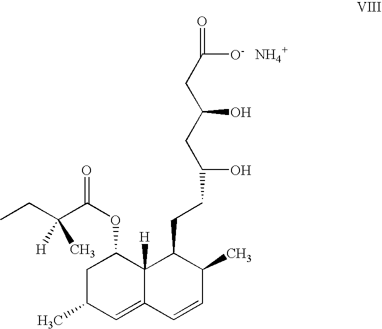

In certain embodiments, the process can further comprise:

(c) converting the lovastatin amide of formula III to simvastatin of formula I.

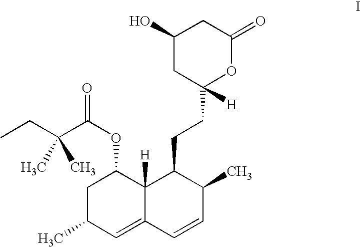

In certain embodiments, the process can further comprise:

(c) combining the lovastatin amide of formula III, a silylation catalyst and hexamethyldisilazane (HMDS) to obtain a third reaction mixture; and

(d) maintaining the third reaction mixture at a temperature and for a period of time sufficient to convert substantially of the lovastatin amide of formula III to the bis (TMS)-lovastatin amide derivative of formula IV; 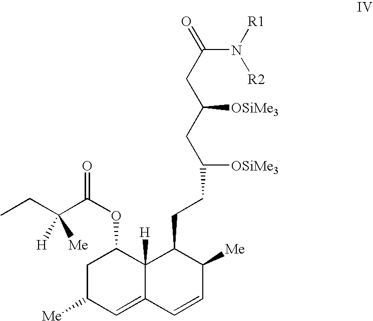
wherein R¹ and R² are as defined previously.

In a certain embodiment, the second reaction mixture comprising the lovastatin amide of formula III of step (b) is directly combined with the silylation catalyst and the HMDS. In such a process, the period of time in step (b) is, e.g., from about 3 to about 5 hours, the period of time in step (d) is from about 0.5 to about 10 hours, the temperature of step (b) is from about 60° to about 120° C. and the temperature of step (d) is from about 0° C. to about 40° C. The molar ratio of the silylation catalyst to the lovastatin compound can be, e.g., from about 0.0001 to about 0.05. The silylation catalyst can be, e.g., silylhalide or iodine and the molar ratio of the silylhalide or iodine to the lovastatin compound can be, e.g., about 0.02 (if silylhalide), or about 0.004 (if iodine). The molar ratio of the HMDS to the lovastatin compound can be, e.g., from about 1 to about 1.7. In a certain embodiment, this process can further comprise:

(e) combining the bis (TMS)-lovastatin amide derivative of formula IV with an aprotic organic solvent to obtain a fourth reaction mixture;

(f) combining the fourth reaction mixture with a strong base at a temperature of from about −10° C. to about −80° C. to obtain a fifth reaction mixture;

(g) maintaining the fifth reaction mixture at a temperature of from about 0° C. to about −40° C. for a period of time of at least about 1 hour (e.g. about 1-5 hours) to obtain a sixth reaction mixture;

(h) combining the sixth reaction mixture with a methylating agent at a temperature of from about 0° C. to about −60° C. to obtain a seventh reaction mixture;

(i) maintaining the seventh reaction mixture at a temperature of from about −20° C. to about −40° C. for a period of time of at least about 0.5 hours (e.g. about 0.5-3 hours) to obtain an eighth reaction mixture;

(j) quenching the eighth reaction mixture at a temperature of from about 0° C. to about −20° C.; and, optionally

(k) recovering simvastatin dihydroxy acid amide derivative of formula V;

wherein R¹ and R² are as defined in formula III.

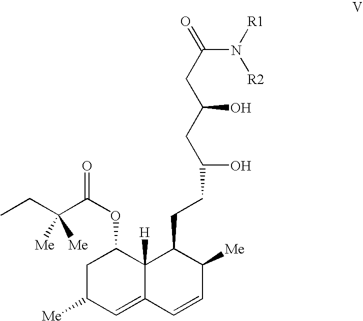

In one embodiment, the above process further comprises:

(l) combining the simvastatin dihydroxy acid amide derivative of formula V, a water miscible organic solvent and an aqueous solution of an alkali base to obtain a ninth reaction mixture; and

(m) maintaining the ninth reaction mixture at a temperature of from about 50° C. to about 100° C. for a period of time of at least about 2 hours (e.g. about 2-8 hours) to obtain an alkali salt of formula IX; 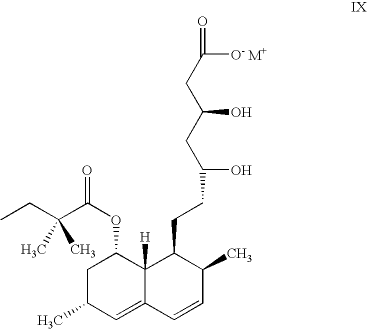
wherein M is alkali metal atom.

In an embodiment, the above process further comprises:

(n) converting the alkali salt of formula IX to simvastatin dihydroxy acid of formula X.

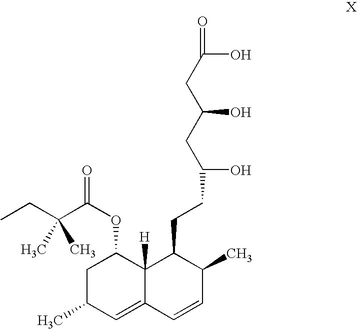

Once the simvastatin dihydroxy acid of formula X is obtained, it may be, e.g., further converted into simvastatin ammonium salt of formula VI, which is then recovered.

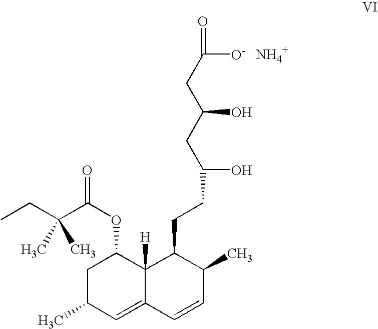

In various embodiments, the simvastatin ammonium salt of formula VI can be converted to simvastatin of formula I.

Certain embodiments relate to a process for the preparation of bis (TMS)-lovastatin amide derivative of formula IV, which may comprise:

(a) combining lovastatin amide of formula III, a silylation catalyst and hexamethyldisilazane (HMDS) to obtain a first reaction mixture; and

(b) obtaining a second reaction mixture comprising the bis (TMS)-lovastatin amide derivative of formula IV by maintaining the first reaction mixture at a temperature and for a period of time sufficient to convert substantially all of the lovastatin amide of formula III to the bis (TMS)-lovastatin amide derivative of formula IV.

The period of time can be, e.g., from about 0.5 to about 10 hours and the temperature can be from about 0° C. to about 40° C. The silylation catalyst can be, e.g., selected from the group consisting of silylhalide, molecular halogen, inorganic salt, organic salt, transition metal phosphonic acid derivative, saccharin and mixtures thereof, e.g., silylhalide, molecular halogen, saccharin or mixtures thereof, e.g., trimethylsilyl iodide, iodine or mixtures thereof.

The molar ratio of the silylation catalyst to the lovastatin amide of formula III can be, e.g., from about 0.0001 to about 0.06. The silylation catalyst can be, e.g., silylhalide and the molar ratio of silylhalide to the lovastatin amide of formula III can be from about 0.02 to about 0.025. The silylation catalyst can be, e.g., iodine and the molar ratio of the iodine to the lovastatin amide of formula III can be from about 0.004 to about 0.005. The molar ratio of the HMDS to the lovastatin amide of formula III can be, e.g., from about 1 to about 2.

If desired, the bis (TMS)-lovastatin amide derivative of formula IV can be converted to simvastatin of formula I using any suitable method.

Certain embodiments relate to a process for the preparation of simvastatin dihydroxy acid amide derivative of formula V, which may comprise:

(a) combining the bis (TMS)-lovastatin amide derivative of formula IV, an aprotic organic solvent, and an amine derivative to obtain a third reaction mixture;

(b) combining the third reaction mixture and a strong base at a temperature of from about −10° C. to about −80° C. to obtain a fourth reaction mixture;

(c) maintaining the fourth reaction mixture at a temperature of from about 0° C. to about −40° C. for a period of time of at least about 1 hour (for example, about 1-5 hours) to obtain a fifth reaction mixture;

(d) combining the fifth reaction mixture with a methylating agent at a temperature of from about 0° C. to about −60° C. to obtain a sixth reaction mixture;

(e) maintaining the sixth reaction mixture at a temperature of from about −20° C. to about −40° C. for a period of time of at least about 0.5 hours (for example, about 0.5-3 hours) to obtain a seventh reaction mixture; and

(f) quenching the seventh reaction mixture at a temperature of from about 0° C. to about −20° C. to obtain the simvastatin dihydroxy acid amide derivative of formula V.

Preferably, the process also includes:

(g) recovering the simvastatin dihydroxy acid amide derivative of formula V.

Any suitable methylating agent may be used. Examples of methylating agents include methyl halide (e.g., methyl iodide, etc.), methyl sulfate and mixtures thereof. The seventh reaction mixture can be quenched, for example, with water. If desired, the simvastatin dihydroxy acid amide derivative of formula V can be converted to simvastatin of formula I.

In certain embodiments, the bis (TMS)-lovastatin amide derivative of formula IV is prepared by a process comprising:

(aa) combining lovastatin amide of formula III, a silylation catalyst and hexamethyldisilazane (HMDS) to obtain a first reaction mixture; and

(bb) obtaining a second reaction mixture comprising the bis (TMS)-lovastatin amide derivative of formula IV by maintaining the first reaction mixture at a temperature and for a period of time sufficient to convert substantially all of the lovastatin amide of formula III to the bis (TMS)-lovastatin amide derivative of formula IV.

The period of time can be, e.g., about 0.5-10 hours and the temperature can be from about 0° C. to about 40° C.

If desired, the second reaction mixture comprising the bis (TMS)-lovastatin amide derivative of formula IV can be directly combined with the aprotic organic solvent and the amide derivative.

The process may further comprise:

(h) combining the simvastatin dihydroxy acid amide derivative of formula V, a water miscible organic solvent and an aqueous solution of an alkali base to obtain an eighth reaction mixture;

(i) maintaining the eighth reaction mixture at a temperature of from about 50° C. to about 100° C. for a period of time of at least about 2 hours (e.g., about 2-8 hours) to obtain an alkali salt of formula IX;

(j) converting the alkali salt of formula IX to simvastatin dihydroxy acid of formula X; and

(k) converting the simvastatin dihydroxy acid of formula X to simvastatin ammonium salt of formula VI.

The process can also comprise:

(l) recovering the simvastatin ammonium salt of formula VI.

The alkali base can be, e.g., sodium hydroxide, potassium hydroxide or mixtures thereof.

The invention also relates to certain novel compounds, which are useful as synthetic intermediates in the preparation of simvastatin. For example, such compounds include a compound of formula IV-a: 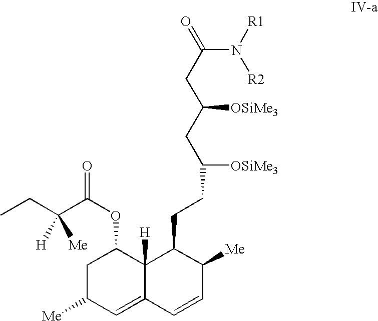
wherein one of R¹ and R² is H and the other of R¹ and R² is selected from the group consisting of benzyl radical and cyclohexyl radical; for example, one of R¹ and R² is H and the other of R¹ and R² is benzyl radical, or one of R¹ and R² is H and the other of R¹ and R² is cyclohexyl radical.

Certain embodiments of the invention also relate to a process for the preparation of simvastatin of formula I, the process comprising:

(a) providing a compound of formula IV-a; and

(b) converting the compound of formula IV-a to simvastatin of formula I.

### DETAILED DESCRIPTION OF THE INVENTION

As used herein, the phrase “at a temperature and for a period of time sufficient to convert substantially all of” a particular starting material to another compound means that at least about 80%, preferably at least about 85%, more preferably at least about 90%, even more preferably at least about 95%, of the starting material is converted to the another compound.

In one embodiment, the invention relates to a synthesis of simvastatin (I) from lovastatin (II) according to the following general scheme. (It will be appreciated that the invention also relates to the individual steps and compounds involved therein).

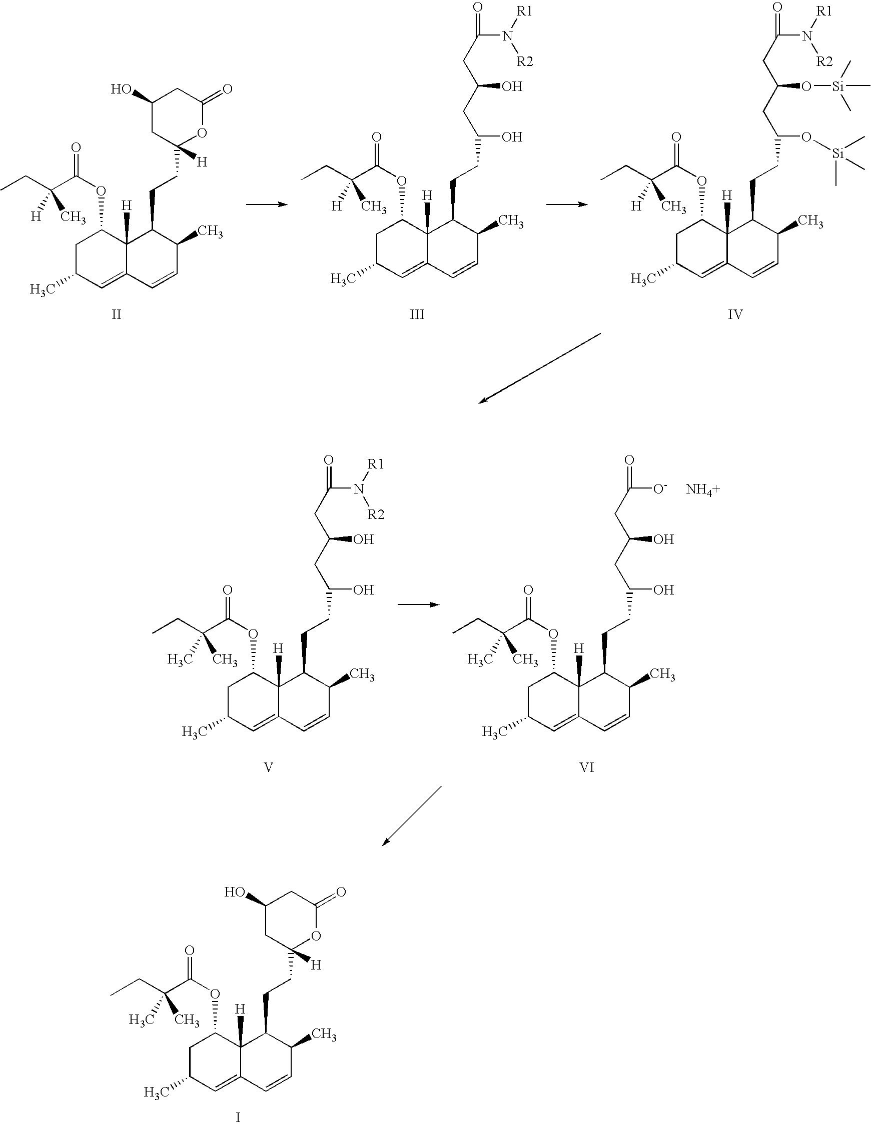

In preferred embodiments, this process affords a simple and economical way for commercial scale production of simvastatin in high yield and purity. In one embodiment, the first four steps of the process may be combined in a “one-pot” process in which the simvastatin ammonium salt (VI) is the first isolated intermediate.

Amidation of Lovastatin

The amidation of lovastatin includes combining a lovastatin compound, an inert organic solvent, and an amine of formula HNR¹R² to obtain a first reaction mixture, which is then converted to a second reaction mixture comprising the lovastatin amide of formula III by maintaining the first reaction mixture at a temperature and for a period of time sufficient to convert substantially all of the lovastatin compound to the lovastatin compound of formula III.

For example, the temperature can be from about 60° C. to about 120° C. and the period of time of can be at least about 3 hours (preferably about 3-5 hours). In certain embodiments, the temperature is from about 80° C. to about 110° C., more preferably from about 80° C. to about 90° C.

Preferably, a Dean-Stark apparatus is used to remove water, which is a by-product.

The lovastatin compound can be selected from the group consisting of

(i) lovastatin of formula II,

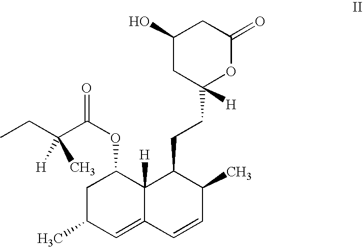

(ii) lovastatin acid of formula VII,

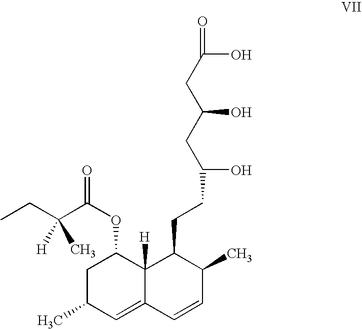

(iii) a salt of lovastatin of formula II,

(iv) a salt of lovastatin acid of formula VII (e.g. the ammonium salt of formula VIII), and

(v) mixtures thereof.

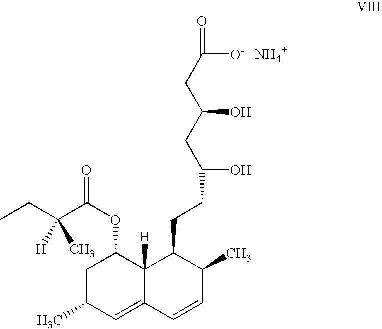

In the amidation reaction, preferably the inert organic solvent is benzene, toluene, xylene, tetrahydrofuran (THF), or mixtures thereof. More preferably, the inert organic solvent is toluene.

The R¹ and R² groups of HNR¹R² are independently selected in each instance from hydrogen, straight or branched C₂₋₈ alkyl, aryl, arylalkyl, and C₃₋₈ cycloalkyl groups, or together form a ring optionally containing a heteroatom such as O, S, or N.

“Aryl”, “aryl group” or “Ar” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7yl, and the like) provided that the point of attachment is through an aromatic ring atom. Preferably, the aryl is phenyl, naphthyl or 5,6,7,8-tetrahydronaphth-2-yl. The aryl may be substituted or unsubstituted. The substituents may be, for example, an alkyl group, an alkenyl group, a cyclic alkyl group, an aralkyl group, a cyclic alkenyl group, a cyano group, an aryl group, an alkoxy group, an aryloxy group, an alkylthio group, an arylthio group, an alkylsulfonyl group, or an arylsulfonyl group.

“Arylalkyl” refers to an aryl group with at least one alkyl substituent, such as a linear or branched alkyl group preferably having from 1 to 10 carbon atoms and more preferably 1 to 6 carbon atoms. The substituent is exemplified by groups such as methyl, t-butyl, n-heptyl, octyl and the like.

Preferably, the amine of formula HNR¹R² is n-butylamine, diethylamine, cyclohexylamine, morpholine, benzylamine, or mixtures thereof. More preferably, the amine of formula HNR¹R² is cyclohexylamine, benzylamine or mixtures thereof. Indeed, the inventor has surprisingly discovered that the amidation reaction works well with amines having relatively large groups—i.e. cyclohexyl and benzyl groups.

The molar ratio of the amine of formula HNR¹R² to the lovastatin compound is no more than about 1.5, more preferably, no more than about 1.2, e.g., a molar ratio of from about 1 to about 1.2 or about 1 to about 1.5. Indeed, the use of no or a slight excess of amine is advantageous, because, e.g., the silylation reaction can be performed without the need to first remove the excess amine by, e.g., distillation, a time-consuming operation that can lead to formation of impurities.

Preferably, the lovastatin of formula II and the amine of formula HNR¹R² are commercially available. Preferably, the lovastatin compound is the ammonium salt of formula VIII.

Preferably, the lovastatin amide of formula III is converted to simvastatin of formula I by a suitable process, e.g., the silylation, methylation, deprotection, and/or lactonization reactions of the general scheme.

Silylation Reaction

In the silylation reaction, the alcohol groups of the lovastatin amide of formula III are protected. In a preferred embodiment, bis(TMS)-lovastatin amide derivative of formula IV is prepared. The silylation reaction is carried out by:

(a) combining lovastatin amide of formula III, a silylation catalyst and hexamethyldisilazane (HMDS) to obtain a reaction mixture; and

(b) maintaining the reaction mixture at a temperature and for a period of time sufficient to convert substantially all of the lovastatin amide of formula III to the bis(TMS)-lovastatin amide of formula IV (wherein R¹ and R² are as defined in formula III):

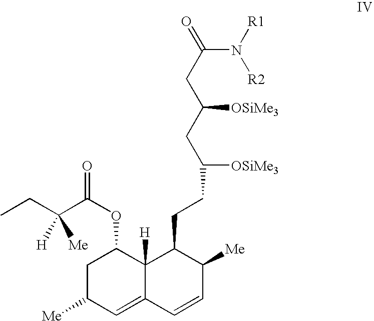

For example, the temperature can be from about 0° C. to about 40° C. (preferably about room temperature to about 40° C.) and the period of time can be at least about 0.5 hours (preferably about 0.5-10 hours, more preferably about 1-4 hours).

Preferably, the silylation catalyst is silylhalide, molecular halogen, inorganic salt, organic salt, transition metal phosphonic acid derivative, saccharin or mixtures thereof. Preferably, the silylhalide is trimethylsilyl iodide, trimethylsilyl bromide, trimethylsilyl chloride, or mixtures thereof, more preferably, trimethylsilyl iodide. Preferably, the molecular halogen is iodine, bromine, or mixtures thereof, more preferably, iodine. Preferably, the inorganic salt is zinc chloride, tetrabutylammonium fluoride, lithium perchlorate, copper triflate, or mixtures thereof. Preferred transition metal phosphonic acid derivative include phosphonomolybdenic acid, tungstenophosphonic acid, and mixtures thereof. The more preferred silylation catalysts are iodine, trimethylsilyl iodide, saccharin and mixtures thereof. The most preferred silylation catalyst is iodine. Surprisingly, the inventor has discovered that silylation catalysts not only improve the rate of reaction, but also decrease the amount of starting material necessary, which is both unexpected and advantageous.

Preferably, the bis(TMS)-lovastatin amide derivative of formula IV is subsequently converted to simvastatin of formula I by a suitable process, e.g., the methylation, deprotection, and/or lactonization steps of the general scheme.

Preferably, the molar ratio of the silylation catalyst to the lovastatin amide of formula III is from about 0.0001 to about 0.06. More preferably, the silylation catalyst is silylhalide and the molar ratio of silylhalide to the lovastatin amide of formula III is from about 0.02 to about 0.025, or the silylation catalyst is iodine and the molar ratio of the iodine to the lovastatin amide of formula III is from about 0.004 to about 0.005. Preferably, the molar ratio of the HMDS to the lovastatin amide of formula III is from about 1 to about 2. Thus, in a preferred embodiment, the silylation reaction of the present invention is performed with no or a slight excess of HMDS. This is advantageous because, for example, 1) HMDS can decompose to ammonia; 2) the excess HMDS does not have to be removed by isolation steps; and 3) isolation steps to remove the excess HMDS can expose the trimethyl-silyl protecting groups to water, which can remove the protecting groups and also lead to insufficient conversion in the methylation step.

Preferably, the lovastatin amide of formula III is prepared by an amidation reaction, e.g., the amidation reaction discussed above. More preferably, the lovastatin of formula III is prepared by the amidation reaction discussed above, and the reaction mixture comprising the amidation reaction product (i.e., the lovastatin amide of formula III) is directly combined with the silylation catalyst and HMDS. That is, the reaction mixture comprising the lovastatin amide of formula III is combined with the silylation catalyst and the HMDS without recovering or purifying the lovastatin amide of formula III from the reaction mixture comprising the lovastatin amide of formula III.

In this preferred embodiment, preferably, the molar ratio of the silylation catalyst to the lovastatin compound is 0.0001 to about 0.05, depending on the catalyst. More preferably, silylhalide is the silylation catalyst and the molar ratio of the silyl halide to the lovastatin compound is about 0.02. More preferably, iodine is the silylation catalyst and the molar ratio of the iodine to the lovastatin compound is about 0.004.

Preferably, the molar ratio of the HMDS to the lovastatin compound is from about 1 to about 1.7, more preferably, from about 1 to about 1.2.

In another aspect, the present invention relates to 2 novel compounds: N-cyclohexyl-7-[1,2,6,7,8,8a(R)-hexahydro-2(S),6(R)-dimethyl-8(S)-[[2(S)-methylbutanoyl]oxy]-1(S)-naphtyl]-3(R),5(R)-bis(trimethylsilyloxy)heptanamide; and N-benzyl-7-[1,2,6,7,8,8a(R)-hexahydro-2(S),6(R)-dimethyl-8(S)-[[2(S)-methylbutanoyl]oxy]-1(S)-naphtyl]-3(R),5(R)-bis(trimethylsilyloxy)heptanamide. These compounds are embraced by formula IV-a:

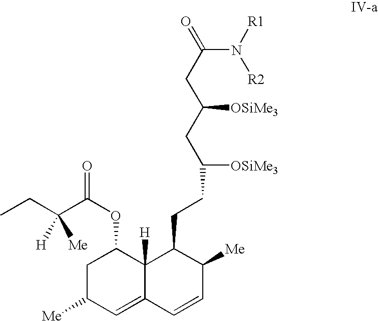

wherein one of R¹ and R² is H and the other of R¹ and R— is benzyl radical or cyclohexyl radical.

The compound of formula IV-a wherein one of R¹ and R² is H and the other of R¹ and R² is cyclohexyl radical can be characterized by data selected from an ¹H-NMR spectrum having hydrogen chemical shifts at about 0.05, 0.73, 0.76, 0.93, 0.97, 1.01-1.09, 1.13, 1.20-1.31, 1.38-1.59, 1.72-1.81, 1.85, 2.05, 2.10, 2.19, 2.25, 2.31, 3.49, 3.63, 4.09, 5.15, 5.37, 5.65, 5.85 and 6.07 ppm, and MS (ESI) spectrum having peaks at about 648.44 (MH⁺).

Meanwhile, the compound of formula IV-a wherein one of R¹ and R² is H and the other of R¹ and R² is benzyl radical can be characterized by data selected from an ¹H-NMR spectrum having hydrogen chemical shifts at about 0.06, 0.11, 0.84, 0.86, 1.05, 1.07, 1.12, 1.14, 1.33, 1.39, 1.52, 1.62-1.64, 1.89, 1.96, 2.19-2.45, 3.58, 4.16, 4.41, 5.31, 5.48, 5.76, 5.95, 6.60 and 7.18-7.33 ppm; and MS (ESI), spectrum having peaks at about 656.42 (MH⁺).

The compounds of formula IV-a can be prepared by any suitable process, e.g., the amidation reaction and silylation reaction described above.

Preferably, the compound of formula IV-a is converted to simvastatin by a suitable process, e.g., the methylation, deprotection and/or lactonization steps of the general scheme.

Methylation Reaction

In one embodiment, a methylation reaction involving the bis(TMS)-lovastatin amide derivative of formula IV can be carried out as follows:

(a) combining the bis(TMS)-lovastatin amide derivative of formula IV with an aprotic organic solvent to obtain a third reaction mixture;

(b) combining the third reaction mixture and a strong base at a temperature of from about −10° C. to about −80° C. to obtain a fourth reaction mixture;

(c) maintaining the fourth reaction mixture at a temperature of from about 0° C. to about −40° C. for a period of time of at least about 1 hour (preferably about 1-5 hours) to obtain a fifth reaction mixture;

(d) combining the fifth reaction mixture with a methylating agent at a temperature of from about 0° C. to about −60° C. to obtain a sixth reaction mixture;

(e) maintaining the sixth reaction mixture at a temperature of from about −20° C. to about −40° C. for a period of time at least about 0.5 hours (preferably about 0.5-3 hours) to obtain a seventh reaction mixture;

(f) quenching the seventh reaction mixture at a temperature of from about 0° C. to about −20° C.; and, optionally,

(g) recovering the simvastatin dihydroxy acid amide derivative of formula V (wherein R¹ and R² are as defined in formula III):

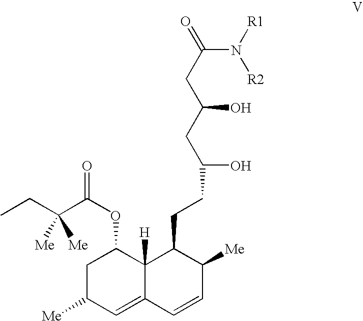

Preferably, the bis(TMS)-lovastatin amide derivative of formula IV is prepared according to the silylation reaction, and the reaction mixture comprising the bis(TMS)-lovastatin amide derivative of formula IV is directly combined with the aprotic organic solvent. That is, the reaction mixture comprising the bis(TMS)-lovastatin amide derivative of formula IV is combined with the aprotic organic solvent without recovering or purifying the bis(TMS)-lovastatin amide derivative of formula IV.

Preferably, the aprotic solvent is aromatic hydrocarbon, ether, or mixtures thereof. Preferably, the aromatic hydrocarbon is toluene. Preferably, the ether is tetrahydrofuran (THF), diethyl ether, diisopropyl ether, dioxane, or mixtures thereof. More preferably, the ether is THF. A preferred mixture is that of toluene and THF.

Preferably, if methylation is preceded by amidation of lovastatin, the amount of the strong base used in the methylation reaction is such that the molar ratio of the strong base to the lovastatin compound is from about 3 to about 6.

Preferably, the strong base is commercially available. More preferably, the strong base is an alkali amide. Preferably, the alkali amide is lithium amide, sodium amide, lithium diethylamide, lithium N-isopropyl-N-cyclohexylamide, lithium diisopropylamide (LDA), lithium pyrrolidide, or mixtures thereof. More preferably, the base is LDA, lithium pyrrolidide or mixtures thereof.

In a particularly preferred embodiment, the strong base is prepared in-situ by adding alkyllithium, alkali hydride, or mixtures thereof to a third reaction mixture that includes, in this embodiment, the bis(TMS)-lovastatin amide derivative and an amine derivative. Preferably, the alkali hydride is sodium hydride, potassium hydride or mixtures thereof. Preferred alkyllithiums include n-butyllithium, n-hexyllithium and mixtures thereof. More preferably, n-butyllithium is used to prepare the strong base. Preferably, the amine derivative is pyrrolidine. Thus, in this preferred embodiment, the alkyllithium, the alkali hydride or mixtures thereof is directly added to the bis(TMS)-lovastatin amide derivative of formula IV, instead of preparing the strong base in a work-up reaction and then combining it with the bis(TMS)-lovastatin amide derivative of formula IV.

Preferably, the temperature of step (b) is from about −30° C. to about −60° C., more preferably, from about −40° C. to about −60° C. Preferably, the temperature of step (c) is from about −30° C. to about −40° C.

Preferably, the methylating agent is methyl halide, methyl sulfate or mixtures thereof. Preferably, the methyl halide is methyl iodide, methyl bromide, methyl chloride or mixtures thereof. A preferred methylsulfate is methyl tosylate, methyl mesylate or mixtures thereof. The more preferred methylating agent is methyl iodide.

Preferably, the methylation reaction is preceded by the amidation reaction described above. In this preferred embodiment, preferably, the amount of the methylating agent used is such that the molar ratio of the methylating agent to the lovastatin compound is from about 1.5 to about 3.

Preferably, the temperature of step of step (d) is from about −30° C. to about −50° C. Preferably, the temperature of step (f) is from about −10° C. to about −20° C.

Preferably, in step (f), the step of quenching the seventh reaction mixture includes the use of water as a quenching reagent. Preferably, the water also removes the silyl groups to produce the desired product, simvastatin dihydroxy acid amide derivative of formula V.

Preferably, simvastatin dihydroxy acid amide derivative of formula V is recovered in step (g) by acidifying the organic phase followed by washing with water and evaporating the solvents.

Preferably, simvastatin dihydroxy acid amide derivative of formula V is subsequently converted to simvastatin by a suitable process, e.g., the deprotection and/or lactonization steps disclosed in the general scheme.

Deprotection Step

In certain embodiments, the amine protecting group of the simvastatin dihydroxy acid amide derivative of formula V can be removed to provide the simvastatin ammonium salt of formula VI. For example, such a process can include:

(a) combining simvastatin dihydroxy acid amide derivative of formula V, a water miscible organic solvent and an aqueous solution of an alkali base to obtain a first reaction mixture;

(b) maintaining the first reaction mixture at a temperature of from about 50° C. to about 100° C. (preferably about 75-80° C.) for a period of time of at least about 2 hours (preferably about 2-8 hours, more preferably 4-8 hours) to obtain second reaction mixture comprising an alkali salt of formula IX;

(c) converting the alkali salt of formula IX to simvastatin dihydroxy acid of formula X; and

(d) converting the simvastatin dihydroxy acid of formula X to simvastatin ammonium salt of formula VI.

Preferably, the process also includes: (e) recovering the simvastatin ammonium salt of formula VI.

Preferably, the simvastatin dihydroxy acid amide derivative of formula V is prepared according to a process set forth previously.

Preferably, the water miscible organic solvent is C₁₋₄ alcohol, ketone, ether or mixtures thereof. Preferred C₁₋₄ alcohols are methanol, ethanol and mixtures thereof. The more preferred water miscible organic solvent is methanol. Preferably, the ketone is acetone. A preferred ethers include THF, dioxane and mixtures thereof.

Preferably, the alkali base is sodium hydroxide, potassium hydroxide or mixtures thereof.

The alkali salt of formula IX can be converted to simvastatin dihydroxy acid of formula X 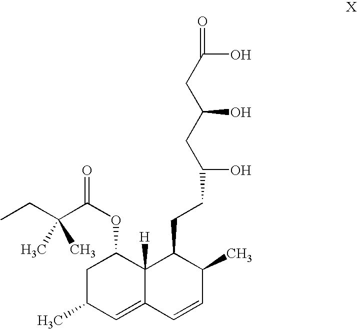
by, e.g., concentrating the second reaction mixture, then adding an acid to adjust the pH of the organic phase to a range of about 2 to about 6, preferably from about 3 to about 5.

Simvastatin dihydroxy acid of formula X can be converted to simvastatin ammonium salt of formula VI 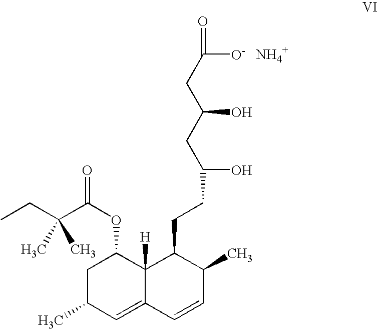
by, e.g., adding ammonium hydroxide to simvastatin dihydroxy acid of formula X.

Simvastatin ammonium salt of formula VI can be recovered by any suitable method, such as cooling to induce precipitation followed by filtering to obtain a wet solid that is then washed.

Lactonization Step

In certain embodiments, the lactonization of simvastatin ammonium salt of formula VI to simvastatin of formula I, may be performed by, for example, a thermally-induced lactonization process, as disclosed in PCT Publication No. WO 2004/071456 A2, which is incorporated herein by reference.

Simvastatin of formula I can be further purified by, e.g., a process involving crystallization from a mixture of an aromatic hydrocarbon and a C₅₋₈ aliphatic hydrocarbon.

### EXAMPLES

### General

In the examples below, the HPLC chromatographic measurements were made on an AGILENT 1100 with a ZORBAX SB C18 4.6*75 mm 3.5 μm, or Hypersil ODS 100*4 mm column, and eluted with a 0.1% aqueous phosphoric acid solution (eluant A)/acetonitrile (eluant B) mixture as described below, with detection at 240 nm, a flow rate of 1.2 mL/min, and an injection volume of 10 μl. The column temperature was 25° C. and the sample temperature was 5° C.

The following gradient program was used with the HPLC:

| Time (min) | eluent A (%) | eluent B (%) |
|---|---|---|
| 0.0 | 50.0 | 50.0 |
| 5.0 | 50.0 | 50.0 |
| 25.0 | 5.0 | 95.0 |
| 34.0 | 5.0 | 95.0 |
| 35.0 | 50.0 | 50.0 |
| 40.0 | 50.0 | 50.0 |

Retention times under these conditions are the following:

| | RT/min | RRT |
|---|---|---|
| Simvastatin hidroxy acid | 5.8 | 0.55 |
| Lovastatin | 8.3 | 0.78 |
| Simvastatin | 10.5 | 1.00 |
| Anhidro-Simvastatin | 16.3 | 1.55 |
| Simvastatin-dimer | 27.3 | 2.59 |

### Example 1

### Preparation of lovastatin cyclohexylamide

Lovastatin (10.1 g, 25 mmol) was suspended in a mixture of cyclohexylamine (2.6 g, 3.0 ml, 26.3 mmol) and toluene (25 ml) and the reaction mixture was heated to a temperature of 80-90° C. to obtain a solution. The solution was stirred at this temperature for 5 hours under nitrogen atmosphere to complete the reaction and obtain a solution including lovastatin cyclohexamide.

### Example 2

### Preparation of bis(TMS)-lovastatin cyclohexylamide

Trimethylsilyl iodide (100 mg, 0.5 mmol) and hexamethyldisilazane (HMDS) (6.03 g, 7.8 ml, 37 mmol) were added to the solution including lovastatin cyclohexamide obtained in Example 1. The resulting reaction mixture was stirred at a temperature of 30-40° C. for 4 hours, to complete the reaction and obtain a reaction mixture including bis(TMS)-lovastatin cyclohexamide.

### Example 3

### Methylation of bis(TMS)-lovastatin cyclohexylamide

The reaction mixture including bis(TMS)-lovastatin cyclohexamide obtained in Example 2 was diluted with THF (100 ml) and cooled to a temperature of −30 to −40° C. Lithium diisopropylamide (60 ml 2 molar solution, 120 mmol) was added to the reaction mixture while stirring at the above temperature under nitrogen. After the addition, the reaction mixture was aged at a temperature of −30 to −35° C. for 1.5 hours. The mixture was then cooled to −50° C. and methyl iodide (8.9 g, 3.8 ml, 62.5 mmol) was added (after which the temperature increased to 14° C.). Then, the reaction mixture was stirred at a temperature of −30 to −35° C. for 1 hour. The temperature was allowed to increase to −10° C. and the reaction mixture was stirred at this temperature for 30 min followed by the addition of water (50 ml). Toluene (50 ml) was added after the water and the organic phase were separated. 1M solution of hydrochloric acid (150 ml) was added to the organic phase to obtain a mixture, which was stirred for 15 min and the phases were separated again. After separation of the aqueous phase, the organic phase was washed twice with water (2×50 ml). The organic phase was concentrated in vacuum to give an oil (about 14 g) containing a simvastatin dihydroxy acid amide derivative.

### Example 4

### Preparation of lovastatin benzylamide

Lovastatin (10.1 g, 25 mmol) was suspended in a mixture of benzylamine (2.94 g, 3 ml, 27.5 mmol) and toluene (25 ml) and the resulting mixture was heated to 80-90° C. to obtain a solution. The solution was stirred at this temperature for 4 hours under nitrogen atmosphere to complete the reaction and obtain a solution including lovastatin benzylamide.

### Example 5

### Preparation of bis(TMS)-lovastatin-benzylamide

Iodine (25 mg, 0.1 mmol) and hexamethyldisilazane (HMDS) (6.03 g, 7.8 ml, 37 mmol) were added to the reaction mixture including lovastatin benzylamide obtained in Example 4. The reaction mixture then was stirred at room temperature for 1 hour. After the 1 hour period, there was no starting material detected by TLC in the reaction mixture.

### Example 6

### Methylation of bis(TMS)-lovastatin benzylamide

The reaction mixture obtained in Example 5 was diluted with THF (50 ml) and toluene (50 ml) followed by addition of pyrrolidine (8.9 g, 10.3 ml; 125 mmol) under nitrogen to give a mixture. The mixture was then cooled to −60° C. A solution of n-butyl lithium (78 ml; 125 mmol) was added over 60 minutes while the temperature was kept at −50 to −60° C. After the addition, the reaction mixture was aged at a temperature of −30 to about −40° C. for 2 hours. The mixture was then cooled to −50 to −60° C., methyl iodide (8.9 g, 3.8 ml; 62.5 mmol) was added over about 10 min at the above temperature, and then the reaction mixture was stirred at −30 to −35° C. for 1 hour. The temperature was allowed to increase to −10° C. and the reaction mixture was stirred at this temperature for 30 min followed by the addition of water (50 ml) to give two phases. After phase separation, 1M solution of sulfuric acid (100 ml) was added to the organic phase and the mixture was stirred for 30 min. Then, the phases were separated again. After separation of the aqueous phase, the organic layer was washed with water (50 ml) and concentrated under vacuum to give an oil (about 15 g) containing a simvastatin dihydroxy acid amide derivative.

### Example 7

### Caustic Hydrolysis and Preparation of Ammonium Salt

The oil of Example 6 was dissolved in methanol (120 ml). Sodium hydroxide (6.4 g, 160 mmol) in water (80 ml) was added to this solution followed by stirring at reflux temperature (75-80° C.) for 4 hours. The obtained solution was then concentrated under vacuum to about half of its original volume. The concentrated mixture was cooled to 5° C. and the pH was adjusted to about 7 by addition of aqueous hydrochloric acid solution. Ethyl acetate (175 ml) was added and the pH was adjusted to 3-5. Then, the water phase was separated and the organic phase was diluted with methanol (50 ml) and the pH was adjusted to 9-11 by adding aqueous ammonia solution (6 ml).

The basic mixture was cooled in a refrigerator and the precipitated material was collected, washed with ethyl acetate and dried to yield simvastatin ammonium salt (9.3 g, 82% yield based on the starting material, lovastatin), in a purity of 97% area by HPLC.

### Example 8

### Preparation of Simvastatin

Simvastatin ammonium salt (6.0 g) in toluene (300 ml) in the presence of butylhydroxytoluene (BHT) (0.08 g) was refluxed for 2 hours, under nitrogen, using a Dean-Stark condenser for removing water. After reflux, the reaction mixture was stirred at 85-90° C. for 3 hours. The reaction mixture was then evaporated to dryness to form a solid residue.

The solid residue was then dissolved in toluene (20 ml) at about 60° C. The solution was treated with charcoal (0.3 g). The charcoal was removed by filtration, and the solution was washed with toluene (4 ml). The solution was then charged into a four-necked round bottomed flask fitted with nitrogen inlet, thermometer, dropping funnel and reflux condenser. The solution was heated to about 60° C. and n-hexane (55 ml) was added in a dropwise manner for 1 hour, while stirring. The reaction mixture was then cooled to 0-5° C. in 1.5 hours and a new portion of hexane (41 ml) was added to the slurry over an hour. The slurry was then stirred at this temperature for another hour and the product was collected, washed with the mixture of toluene (4 ml) and hexane (16 ml) containing BHT (butylated hydroxytoluene) (0.007 g) and dried at 48° C. in a vacuum oven to yield simvastatin (5.0 g, 90% yield, based on the starting material, simvastatin ammonium salt) in a purity of 98% (HPLC).

### Example 9

### Preparation of lovastatin benzylamide

Lovastatin ammonium salt (11.0 g, 25 mmol) was suspended in a mixture of benzylamine (3.2 g, 3.3 ml, 30 mmol) and toluene (30 ml) and the mixture was heated to reflux temperature. The mixture was stirred at reflux temperature for 3 hours under nitrogen atmosphere using a Dean-Stark water separator to complete the formation of the lovastatin benzylamide.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the invention.

1. A process for preparing lovastatin amide of formula III, 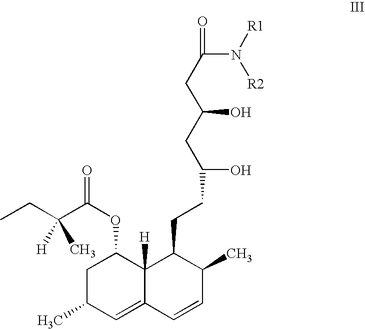
the process comprising:
(a) reacting a lovastatin compound selected from the group consisting of (i) lovastatin of formula II, 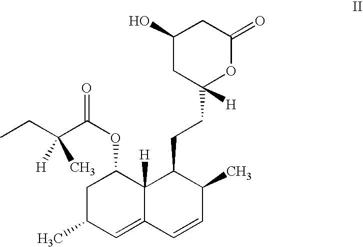
(ii) lovastatin acid of formula VII, 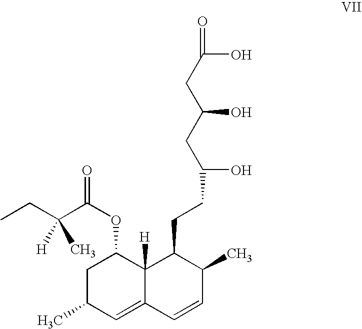
(iii) a salt of lovastatin of formula II,
(iv) a salt of lovastatin acid of formula VII, and
(v) mixtures thereof,
with an amine of formula HNR¹R² to form the lovastatin amide of formula III;
wherein R¹ and R² are independently selected from hydrogen, straight or branched C₂₋₈ alkyl, aryl, arylalkyl, and C₃₋₈ cycloalkyl groups, or together form a ring optionally containing a heteroatom; and
wherein the molar ratio of the amine of formula HNR¹R² to the lovastatin compound is no more than about 1.5.

2. (canceled)

3. (canceled)

4. (canceled)

5. (canceled)

6. (canceled)

7. (canceled)

8. A process for preparing simvastatin of formula I comprising:
(a) preparing lovastatin amide of formula III according to the process of claim 1; and
(b) converting the lovastatin amide of formula III to simvastatin of formula I

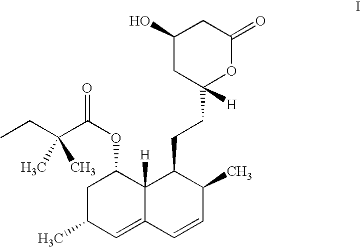

9. A process for preparing bis(TMS)-lovastatin amide derivative of formula IV comprising:
(a) preparing lovastatin amide of formula III according to the process of claim 1; and
(b) reacting the lovastatin amide of formula III with hexamethyldisilazane (HMDS) in the presence of a silylation catalyst to form the bis(TMS)-lovastatin amide derivative of formula IV; 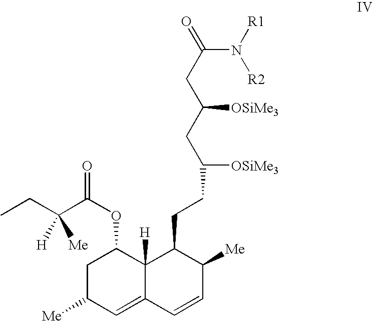
wherein R¹ and R² are as defined in claim 1.

10. The process of claim 9 wherein the lovastatin amide of formula III is directly combined with the silylation catalyst and the HMDS.

11. (canceled)

12. The process of claim 9 wherein the molar ratio of the silylation catalyst to the lovastatin compound is from about 0.0001 to about 0.05.

13. (canceled)

14. (canceled)

15. The process of claim 9 wherein the molar ratio of the HMDS to the lovastatin compound is from about 1 to about 1.7.

16. A process for preparing simvastatin dihydroxy acid amide derivative of formula V comprising:
(a) preparing bis(TMS)-lovastatin amide derivative of formula IV according to the process of claim 9;
(b) combining the bis(TMS)-lovastatin amide derivative of formula IV with an aprotic organic solvent to obtain a first reaction mixture;
(c) combining the first reaction mixture with a strong base at a temperature of from about −10° C. to about −80° C. to obtain a second reaction mixture;
(d) maintaining the second reaction mixture at a temperature of from about 0° C. to about −40° C. for a period of time of at least about 1 hour to obtain a third reaction mixture;
(e) combining the third reaction mixture with a methylating agent at a temperature of from about 0° C. to about −60° C. to obtain a fourth reaction mixture;
(f) maintaining the fourth reaction mixture at a temperature of from about −20° C. to about −40° C. for a period of time of at least about 0.5 hours to obtain a fifth reaction mixture;
(g) quenching the fifth reaction mixture at a temperature of from about 0° C. to about −20° C.; and, optionally,
(h) recovering simvastatin dihydroxy acid amide derivative of formula V;
wherein R¹ and R² are as defined in claim 1

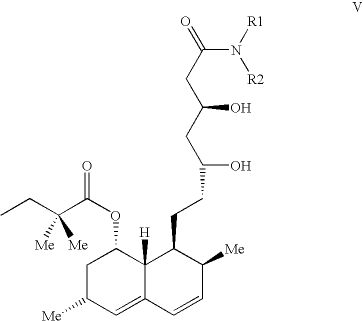

17. A process for preparing an alkali salt of formula IX comprising:
(a) preparing simvastatin dihydroxy acid amide derivative of formula V according to the process of claim 16;
(b) combining the simvastatin dihydroxy acid amide derivative of formula V, a water miscible organic solvent and an aqueous solution of an alkali base to obtain a sixth reaction mixture; and
(c) maintaining the sixth reaction mixture at a temperature of from about 50° C. to about 100° C. for a period of time of at least about 2 hours to obtain an alkali salt of formula IX;
wherein M is alkali metal atom

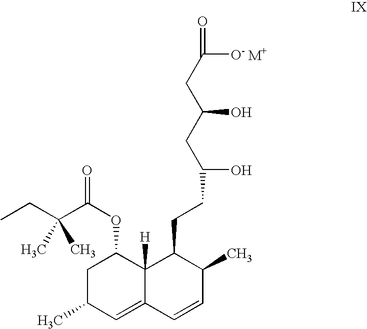

18. A process for preparing simvastatin dihydroxy acid of formula X comprising:
(a) preparing alkali salt of formula IX according to the process of claim 17; and
(b) converting the alkali salt of formula IX to simvastatin dihydroxy acid of formula X

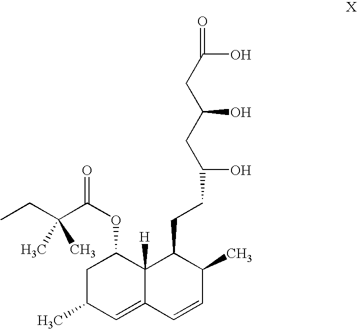

19. A process for preparing simvastatin ammonium salt of formula VI comprising:
(a) preparing simvastatin dihydroxy acid of formula X according to the process of claim 18;
(b) converting the simvastatin dihydroxy acid of formula X to simvastatin ammonium salt of formula VI; and
(c) recovering the simvastatin ammonium salt of formula VI

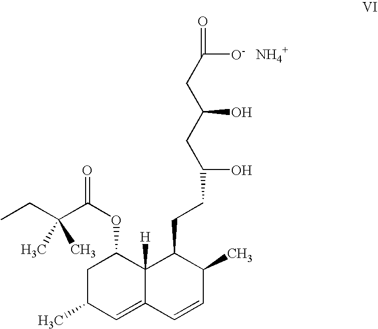

20. A process for preparing simvastatin of formula I comprising:
(a) preparing simvastatin ammonium salt of formula VI according to the process of claim 19; and
(b) converting the simvastatin ammonium salt of formula VI to simvastatin of formula I.

21. (canceled)

22. A process for the preparation of bis(TMS)-lovastatin amide derivative of formula IV, the process comprising:
(a) reacting lovastatin amide of formula III with hexamethyldisilazane (HMDS) in the presence of a silylation catalyst to form the bis(TMS)-lovastatin amide derivative of formula IV.

23. (canceled)

24. The process of claim 22 wherein the silylation catalyst is selected from the group consisting of silylhalide, molecular halogen, inorganic salt, organic salt, transition metal phosphonic acid derivative, saccharin and mixtures thereof.

25. (canceled)

26. (canceled)

27. The process of claim 22 wherein the molar ratio of the silylation catalyst to the lovastatin amide of formula III is from about 0.0001 to about 0.06.

28. (canceled)

29. (canceled)

30. The process of claim 22 wherein the molar ratio of the HMDS to the lovastatin amide of formula III is from about 1 to about 2.

31. A process for preparing simvastatin of formula I comprising:
(a) preparing bis(TMS)-lovastatin amide derivative of formula IV according to the process of claim 22; and
(b) converting the bis(TMS)-lovastatin amide derivative of formula IV to simvastatin of formula I.

32. A process for the preparation of simvastatin dihydroxy acid amide derivative of formula V, comprising:
(a) combining a bis(TMS)-lovastatin amide derivative of formula IV, an aprotic organic solvent, an amine derivative to obtain a third reaction mixture;
(b) combining the third reaction mixture and a compound selected from the group consisting of alkyllithium, alkali hydride, and mixtures thereof at a temperature of from about −10° C. to about −80° C. to obtain a fourth reaction mixture;
(c) maintaining the fourth reaction mixture at a temperature of from about 0° C. to about −40° C. for a period of time of at least about 1 hour to obtain a fifth reaction mixture;
(d) combining the fifth reaction mixture with a methylating agent at a temperature of from about 0° C. to about −60° C. to obtain a sixth reaction mixture;
(e) maintaining the sixth reaction mixture at a temperature of from about −20° C. to about −40° C. for a period of time at least about 0.5 hours to obtain a seventh reaction mixture; and
(f) quenching the seventh reaction mixture at a temperature of from about 0° C. to about −20° C. to obtain the simvastatin dihydroxy acid amide derivative of formula V.

33. The process of claim 32 further comprising:
(g) recovering the simvastatin dihydroxy acid amide derivative of formula V.

34. (canceled)

35. (canceled)

36. (canceled)

37. A process for preparing simvastatin of formula I comprising:
(a) preparing simvastatin dihydroxy acid amide derivative of formula V according to the process of claim 32; and
(b) converting the simvastatin dihydroxy acid amide derivative of formula V to simvastatin of formula I.

38. The process of claim 32 further comprising preparing the bis(TMS)-lovastatin amide derivative of formula IV by a process comprising:
(aa) reacting lovastatin amide of formula III with hexamethyldisilazane (HMDS) in the presence of a silylation catalyst to form the bis(TMS)-lovastatin amide derivative of formula IV.

39. (canceled)

40. The process of claim 38 wherein the bis(TMS)-lovastatin amide derivative of formula IV is directly combined with the aprotic organic solvent and the amine derivative.

41. A process for preparing simvastatin ammonium salt of formula VI comprising:
(a) preparing simvastatin dihydroxy acid amide derivative of formula V according to the process of claim 32;
(b) combining the simvastatin dihydroxy acid amide derivative of formula V, a water miscible organic solvent and an aqueous solution of an alkali base to obtain an eighth reaction mixture;
(c) maintaining the eighth reaction mixture at a temperature of from about 50° C. to about 100° C. for a period of time of at least about 2 hours to obtain an alkali salt of formula IX;
(d) converting the alkali salt of formula IX to simvastatin dihydroxy acid of formula X; and
(e) converting the simvastatin dihydroxy acid of formula X to simvastatin ammonium salt of formula VI.

42. The process of claim 41 further comprising
(l) recovering the simvastatin ammonium salt of formula VI.

43. (canceled)

44. (canceled)

45. A compound of formula IV-a: 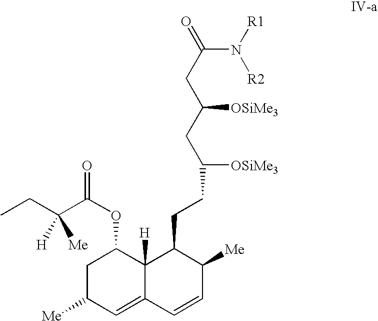
wherein one of R¹ and R² is H and the other of R¹ and R² is selected from the group consisting of benzyl radical and cyclohexyl radical.

46. (canceled)

47. (canceled)

48. A process for the preparation of simvastatin of formula I, the process comprising:
(a) providing the compound of claim 45; and
(b) converting the compound of claim 45 to simvastatin of formula I.
